(19) United States
(12) Patent Application Publication
(10) Pub. No.: US 2004/0203138 A1
(43) Pub. Date: Oct. 14, 2004

(54) Polynucleotide array fabrication

(76) Inventors: Michael P. Caren, Palo Alto, CA (US); Herbert F. Cattell, Mountain View, CA (US); Richard P. Tella, Sunnyvale, CA (US); Peter G. Webb, Menlo Park, CA (US); Jay K. Bass, Mountain View, CA (US)

Correspondence Address: AGILENT TECHNOLOGIES, INC., INTELLECTUAL PROPERTY ADMINISTRATION, LEGAL DEPT., P.O. BOX 7599, M/S DL429, LOVELAND, CO, 80537-0599, US

(21) Appl. No.: 10/833,607

(22) Filed: Apr. 27, 2004

Related U.S. Application Data
(63) Continuation of application No. 09/302,898, filed on Apr. 30, 1999.

(51) Int. Cl. B05D 3/00; C12Q 1/68; C07H 21/04; C12M 1/34

(52) U.S. Cl. 435/287.2; 427/2.11

(57) ABSTRACT

A method and apparatus for fabricating an array of polynucleotides on a substrate. A polynucleotide deposition system is operated to deposit an array of polynucleotide containing fluid droplets on the substrate and which, when dry, will yield polynucleotide containing spots of respective target locations and dimensions. Droplets deposited by the system are allowed to dry to yield actual spots. An image is captured of the substrate with dried actual spots. Dried actual spot locations or dimensions from the image, are compared with target locations or dimensions of polynucleotide containing spots. A signal indicative of the result of the comparison may be generated.

### CROSS-REFERENCE TO RELATED APPLICATIONS

This application is a continuation of application Ser. No. 09/302,898 filed on Apr. 30, 1999; the disclosure of which is herein incorporated by reference.

### FIELD OF THE INVENTION

This invention relates to arrays, particularly polynucleotide arrays such as DNA arrays, which are useful in diagnostic, screening, gene expression analysis, and other applications.

### BACKGROUND OF THE INVENTION

Polynucleotide arrays (such as DNA or RNA arrays), are known and are used, for example, as diagnostic or screening tools. Such arrays include regions (sometimes referenced as spots or features) of usually different sequence polynucleotides arranged in a predetermined configuration on a substrate. The arrays, when exposed to a sample, will exhibit an observed binding pattern. This binding pattern can be detected, for example, by labeling all polynucleotide targets (for example, DNA) in the sample with a suitable label (such as a fluorescent compound), and accurately observing the fluorescence pattern on the array. Assuming that the different sequence polynucleotides were correctly deposited in accordance with the predetermined configuration, then the observed binding pattern will be indicative of the presence and/or concentration of one or more polynucleotide components of the sample.

Biopolymer arrays can be fabricated using either in situ synthesis methods or deposition of the previously obtained biopolymers. The in situ synthesis methods include those described in U.S. Pat. No. 5,449,754 for synthesizing peptide arrays, as well as WO 98/41531 and the references cited therein for synthesizing polynucleotides (specifically, DNA). Such in situ synthesis methods can be basically regarded as iterating the sequence of depositing droplets of: (a) a protected monomer onto predetermined locations on a substrate to link with either a suitably activated substrate surface (or with a previously deposited deprotected monomer); (b) deprotecting the deposited monomer so that it can now react with a subsequently deposited protected monomer; and (c) depositing another protected monomer for linking. Different monomers may be deposited at different regions on the substrate during any one iteration so that the different regions of the completed array will have different desired biopolymer sequences. One or more intermediate further steps may be required in each iteration, such as oxidation and washing steps. The deposition methods basically involve depositing biopolymers at predetermined locations on a substrate which are suitably activated such that the biopolymers can link thereto. Biopolymers of different sequence may be deposited at different regions of the substrate to yield the completed array. Washing or other additional steps may also be used.

Typical procedures known in the art for deposition of polynucleotides, particularly DNA such as whole oligomers or cDNA, are to load a small volume of DNA in solution in one or more drop dispensers such as the tip of a pin or in an open capillary and, touch the pin or capillary to the surface of the substrate. Such a procedure is described in U.S. Pat. No. 5,807,522. When the fluid touches the surface, some of the fluid is transferred. The pin or capillary must be washed prior to picking up the next type of DNA for spotting onto the array. This process is repeated for many different sequences and, eventually, the desired array is formed. Alternatively, the DNA can be loaded into a drop dispenser in the form of an inkjet head and fired onto the substrate. Such a technique has been described, for example, in PCT publications WO 95/25116 and WO 98/41531, and elsewhere. This method has the advantage of non-contact deposition. Still other methods include pipetting and positive displacement pumps such as the Biodot equipment (available from Bio-Dot Inc., Irvine Calif., USA).

In array fabrication, the quantities of DNA available for the array are usually very small and expensive. Sample quantities available for testing are usually also very small and it is therefore desirable to simultaneously test the same sample against a large number of different probes on an array. These conditions require use of arrays with large numbers of very small, closely spaced spots. It is important in such arrays that spots actually be present, that they are put down accurately in the desired pattern, are of the correct size, and that the DNA is uniformly coated within the spot.

It would be useful then, to be able to fabricate arrays such that spot errors can be readily detected. It would also be useful if, when errors are present, they can be quantified in some aspect (so that they can be compensated for during use of the array, for example). It would further be useful if errors which might have occurred even following droplet deposition, could be detected and/or quantified.

### SUMMARY OF THE INVENTION

The present invention realizes that many factors can lead to spot position errors or other spot errors. For example, small displacements in expected drop dispenser positions relative to the substrate during drop dispensing, can result from manufacturing tolerances or vibrations. Also, one or more dispensers may malfunction at some time during their lifetime and dispense an abnormally small drop or no drop. Further, the present invention also realizes that even drops correctly deposited at target locations may move from those locations before they have completely dried, due to vibration and possibly variations in substrate surface hydrophobicity and other factors. Any method which only evaluates locations of droplets immediately after deposition, could therefore fail to detect the actual final locations of the dried spots. Additionally, the present invention also recognizes that it is possible that an operator failed to provide the polynucleotide (particularly DNA) in the required solution. Also, in cases where the polynucleotide is made by an amplification reaction (such as the well known PCR amplification technique) the technique can, on occasion fail for various reasons, and a separate analysis step would normally be required to confirm success. Use of a method which only observes locations of droplets immediately after deposition would not provide any convenient indication of such operator or reaction failure.

The present invention then, provides a method for fabricating an array of polynucleotides on a substrate. The method includes depositing an array of polynucleotide containing fluid droplets on the substrate to provide, when dry, a target pattern of polynucleotide containing dried spots. Any device or apparatus which can be used to deposit droplets in an array can be used as a deposition system to accomplish this. The target pattern then, is an aim or desired pattern. A sufficient time is allowed to pass such that droplets deposited by the system will have dried to yield an actual pattern of dried spots. The actual pattern is then observed. That is, at least one characteristic (such as the presence of dried spots at particular locations) of the actual pattern is determined, such as by capturing an image of the substrate with dried actual spots. The actual pattern is compared with the target pattern. By this is referenced that the determined characteristic of the actual pattern is compared with the corresponding characteristic of the target pattern (for example, the actual presence or absence of dried spots at particular locations, is compared with the target locations). A signal may be generated which is indicative of a result of the comparison. The target and actual patterns may particularly include target locations and dimensions, and the pattern comparison may include comparing dried actual spot locations or dimensions from the image, with target locations or dimensions of polynucleotide containing spots.

At least some of the fluid droplets will typically contain respective different polynucleotides. One or more of the polynucleotide fluids may also contain a salt. A sufficient amount of the salt is present to enhance imaging of the polynucleotide. That is, it is easier to distinguish the presence or absence of a polynucleotide in a dried spot, when the salt is present. Presence of the salt, particularly when the polynucleotide is DNA, facilitates identification of potential polynucleotide fluid errors (such as the absence of any DNA due to operator or reaction failure). The polynucleotides may be at least six or ten nucleotides in length, or even at least one hundred or one thousand nucleotides in length. The polynucleotides may be RNA, DNA (for example, cDNA) or contain a synthetic backbone as mentioned below, and while they will typically be single stranded, can also include double stranded polynucleotides. During image capture any of a number of characteristics of dried spots may be imaged. For example, light scattering characteristics of dried spots may be imaged such as by using visible or other light, or fluorescence characteristics of dried spots may be imaged.

In a typical operation, the deposition system is operated to fabricate multiple polynucleotide arrays on different substrates or on a same substrate. The present invention also contemplates, when the results of one or more comparisons for an array exceed a predetermined tolerance, storing an error indication in association with that array. An error indication (sometimes referenced as “error data”) may simply be an indication of some error (for example, that a particular spot is mis-positioned) or could include an indication of the magnitude of the error (for example, the actual location of a mis-positioned spot). This error indication can be used in a number of ways. For example, it may be used to reject the associated array. In this way, a low error rate is maintained in arrays eventually provided to end users. The error indication could be written on a medium, and the medium physically associated with the array. Alternatively, only an identification of the associated array could be provided to an end-user. This can be done by writing an identification of the error on a medium (in human and/or machine readable characters) and physically associating the medium with the array. The identification would also be stored in a memory with the corresponding error indication. In this manner, a user of the array could later retrieve the error indication from the memory using the written identification on the medium associated with the array. Additionally, or alternatively, the method can additionally include, when the results of one or more comparisons for an array exceed a predetermined tolerance indicating an error condition, automatically halting further operation of the deposition system and generating a visible or audible operator alert. This can allow for operator inspection and correction of the error source, and can avoid reproducing more arrays with unacceptable errors. Alternatively or additionally, this also can allow correcting at least some types of errors on arrays already fabricated (for example, if a given pulse jet has failed to fire or mis-fired, another pulse jet may be used to correctly deposit a droplet).

In the case where the fluid dispensing head has multiple drop dispensers, and multiple error indications are generated (either for a same array or for different arrays), the method can additionally include evaluating if a same drop dispenser is responsible. If the evaluation result indicates the same drop dispenser may be responsible, a visible (such as on a CRT) or audible (such as voice synthesized) operator alert can be generated which includes an indication of the responsible drop dispenser. This indication can, for example, be a direct indication of the responsible drop dispenser (for example, in the form of the physical location of the responsible drop dispenser). An operator can use this information, for example, to evaluate whether the head needs replacing or to check whether a solution preselected to be dispensed by that dispenser is in error (for example, by the polynucleotide concentration being substantially incorrect, including the possibility of no polynucleotide being present). Alternatively, it may be an indirect indication by suggesting that the preselected solution to be dispensed by that dispenser may be in error.

In the case where the dispensing head has multiple drop dispensers and the deposition system includes a control processor, the control processor may direct loading of the dispensers in a pattern in which at least some of the dispensers are loaded with the same fluid. For example, each set of two or six dispensers on a head with multiple such sets, could be loaded with the same fluid. In this situation, when multiple error indications are generated, the control processor compares a pattern of error indications with the loading pattern of the dispensers. From this, the processor can evaluate whether one or more drop dispensers or an error in a polynucleotide containing fluid is responsible for the error indications. For example, if the processor determines that there are repeated errors from the same drop dispenser of a set loaded with the same fluid while not from other members of the set, this can be taken as an indication that there is a potential drop dispenser error in the form of a malfunction of the particular drop dispenser. On the other hand, if there are repeated errors from all members of a set loaded with the same fluid, this can be taken as an indication that there is a potential error in the fluid (for example, it does not contain polynucleotide of the expected concentration).

When an evaluation of multiple error indications indicates that a same drop dispenser in a multiple drop dispenser head may be responsible (that is, it is suspect), the method may include altering an initial deposition pattern from the head (such as may have been formulated or accessed by a control processor) such that the suspect drop dispenser is not used. The target array pattern can still be obtained by using the another dispenser in the head to perform the deposition previously required by the suspect dispenser, whether during a same pass over the substrate on which the suspect dispenser would have dispensed droplets, or whether or an additional pass.

The present invention further provides apparatus which can execute any of the methods of the present invention. In one aspect, an apparatus of the present invention for fabricating an array of polynucleotides on a substrate, includes a polynucleotide deposition system as already mentioned. An imaging system is provided to capture the image of the actual pattern. An imaging system can include any system which can provide spatial information as to the location of dried drops. A processor controls the deposition system to deposit the array of droplets and, after a predetermined time has elapsed for drying of the droplets to yield the actual pattern, causes the imaging system to capture an image of the actual pattern. The processor executes the comparison of the actual and target patterns. The deposition system may include a head having multiple jets each of which can dispense droplets of a fluid onto a substrate. Each jet includes a chamber with an orifice, and includes an ejector which, when activated, causes a droplet to be ejected from the orifice.

The processor may be configured to cause the remainder of the apparatus to execute any of the steps required by the any of the methods of the present invention. These include any of: operating the deposition system to deposit multiple polynucleotide arrays; causing the imaging system to capture one or more images of such arrays; performing the comparison step for such arrays; operating the deposition system to correct for any detected errors; automatically halting further operation of the deposition system upon multiple error indications; generating any of the operator alerts on the output device; evaluating drop dispenser and polynucleotide containing fluid errors mentioned above; and altering the initial dispensing pattern.

The present invention further provides a kit having a substrate carrying an array of biological moieties, such as polynucleotides. The kit also includes a medium carrying error data describing one or more errors in the array. The medium may particularly be a machine readable medium (such as a computer readable optical or magnetic disk, tape or other medium).

Apparatus and methods of the present invention can optionally be used to fabricate arrays of other moieties, such as nucleotide monomers (as may be used in the in situ process for forming polynucleotide arrays) or proteins. Furthermore, the error indication and any subsequent steps acting on one or more error indications (including correcting by a remote user), may alternatively be used with other means of detecting spot location (such as imaging deposited liquid droplets). However, for reasons discussed herein, it is preferred that one or more images of actual dried spots be used.

The method, apparatus, and kits of the present invention can provide any one or more of a number of useful benefits. For example, if an error (such as no spot deposition or a spot placement error) is found, the deposition system can re-work the array during the manufacturing process (for example, by using another jet to deposit a spot at a location where an error in the form of no spot was found). Also, when an array is manufactured with an error (such as spot location or polynucleotide concentration error), this can be identified by the present methods and apparatus, sufficiently well such that its presence can be compensated for during manufacture or use of the array if desired. Polynucleotide containing solutions which also contain a salt (such as a buffer) are particularly readily distinguished from such solutions in which no or little polynucleotide is present, further aiding in evaluating the presence and type of error.

### BRIEF DESCRIPTION OF THE DRAWINGS

FIG. 1 is a perspective view of a substrate bearing multiple arrays, as may be produced by a method and apparatus of the present invention;

FIG. 2 is an enlarged view of a portion of FIG. 1 showing some of the identifiable individual regions of a single array of FIG. 1;

FIG. 3 is an enlarged cross-section of a portion of FIG. 2;

FIG. 4 is a schematic view of apparatus of the present invention;

FIG. 5 is an enlarged cross-section of a loading station of the apparatus of FIG. 4;

FIGS. 6-8 illustrate various arrangements on imaging system components in the apparatus of FIG. 4;

FIG. 9 is an enlarged schematic plan view of dried spots of an array to illustrate how pattern evaluation can provide an indication of errors;

FIGS. 10-13 are enlarged photographs of dried spots of actual arrays with various DNA concentrations; and

FIG. 14 is a photograph similar to that of FIGS. 10-13 and illustrating the effect of having a salt present.

To facilitate understanding, identical reference numerals have been used, where practical, to designate identical elements that are common to the figures.

### DETAILED DESCRIPTION OF EMBODIMENTS OF THE INVENTION

In the present application, unless a contrary intention appears, the following terms refer to the indicated characteristics. A “biopolymer” is a polymer of one or more types of repeating units. Biopolymers are found in biological systems and particularly include peptides or polynucleotides, as well as such compounds composed of or containing amino acid or nucleotide analogs or non-nucleotide groups. This includes polynucleotides in which the conventional backbone has been replaced with a non-naturally occurring or synthetic backbone, and nucleic acids in which one or more of the conventional bases has been replaced with a synthetic base capable of participating in Watson-Crick type hydrogen bonding interactions. Polynucleotides include single or multiple stranded configurations, where one or more of the strands may or may not be completely aligned with another. A “nucleotide” refers to a subunit of a nucleic acid and has a phosphate group, a 5 carbon sugar and a nitrogen containing base, as well as analogs of such subunits. Specifically, a “biopolymer” includes DNA (including cDNA), RNA and oligonucleotides. An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (for example, a single amino acid or nucleotide with two linking groups one or both of which may have removable protecting groups). A biomonomer fluid or biopolymer fluid reference a liquid containing either a biomonomer or biopolymer, respectively (typically in solution). An “array”, unless a contrary intention appears, includes any one or two dimensional arrangement of discrete regions bearing particular biopolymer moieties (for example, different polynucleotide sequences) associated with that region. It will also be appreciated that throughout the present application, words such as “upper”, “lower” and the are used with reference to a particular orientation of the apparatus with respect to gravity, but it will be understood that other operating orientations of the apparatus or any of its components, with respect to gravity, are possible. Reference to a “droplet” being dispensed from a pulse jet herein, merely refers to a discrete small quantity of fluid (usually less than about 1000 pL) being dispensed upon a single pulse of the pulse jet (corresponding to a single activation of an ejector) and does not require any particular shape of this discrete quantity. When a “spot” is referred to, this may reference a dried spot on the substrate resulting from drying of a dispensed droplet, or a wet spot on the substrate resulting from a dispensed droplet which has not yet dried, depending upon the context. “Fluid” is used herein to reference a liquid. By one item being “remote” from another is referenced that they are at least in different buildings, and may be at least one, at least ten, or at least one hundred miles apart.

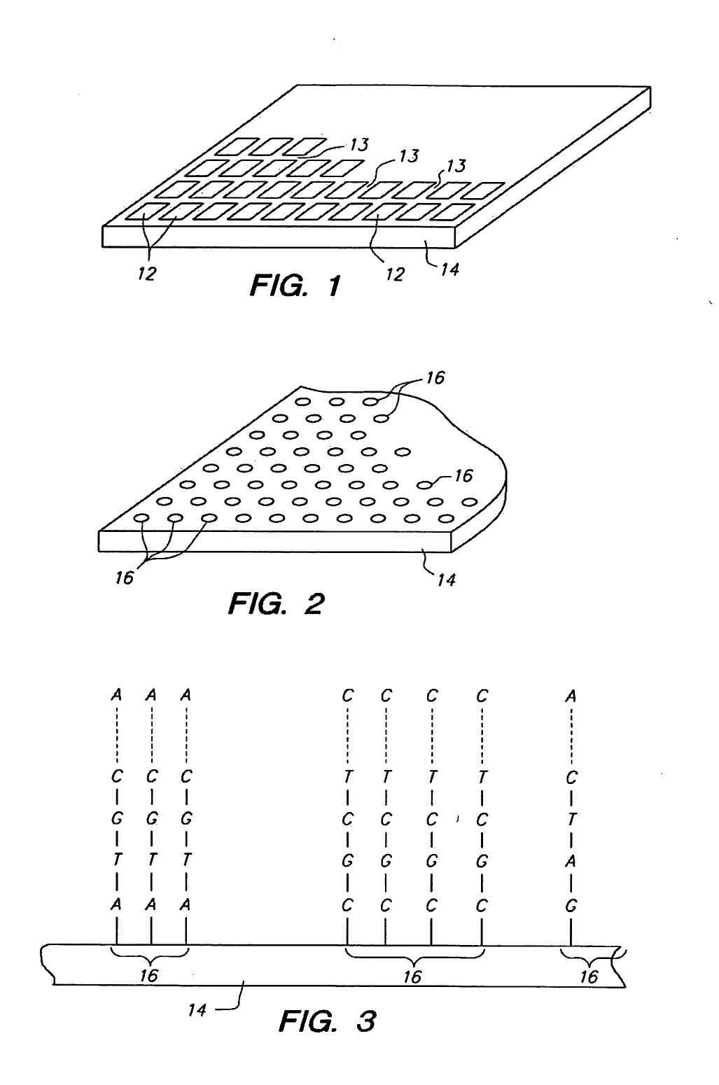

Referring first to FIGS. 1-3, typically the present invention will produce multiple identical arrays 12 (only some of which are shown in FIG. 1) across the complete surface of a single substrate 14. However, the arrays 12 produced on a given substrate need not be identical and some or all could be different. Each array 12 will contain multiple spots or regions 16. A typical array 12 may contain from 100 to 100,000 regions. All of the regions 16 may be different, or some or all could be the same. Each region carries a predetermined polynucleotide having a particular sequence, or a predetermined mixture of polynucleotides. This is illustrated schematically in FIG. 3 where regions 16 are shown as carrying different polynucleotide sequences.

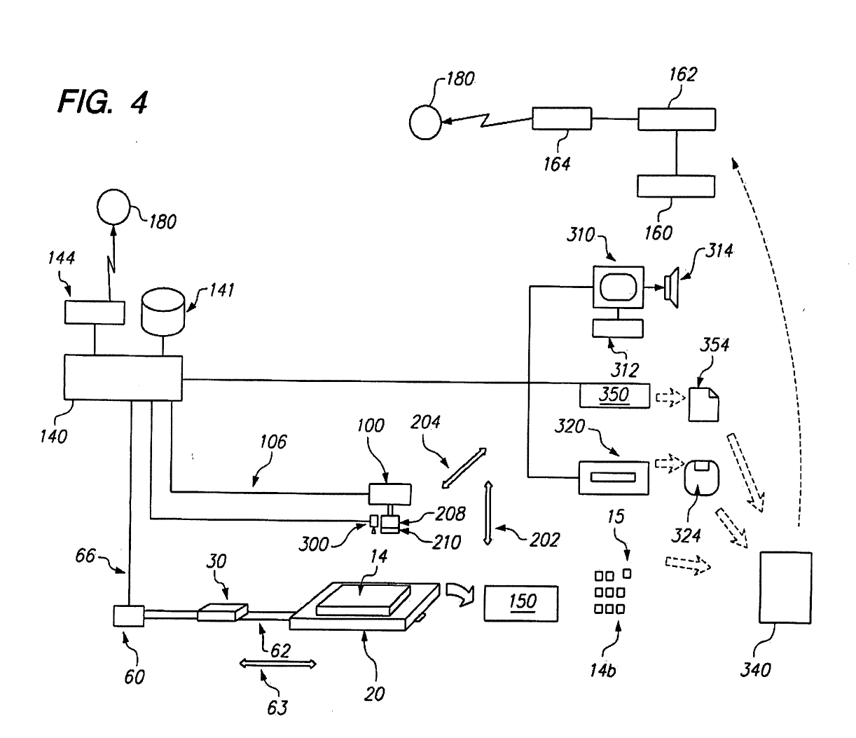

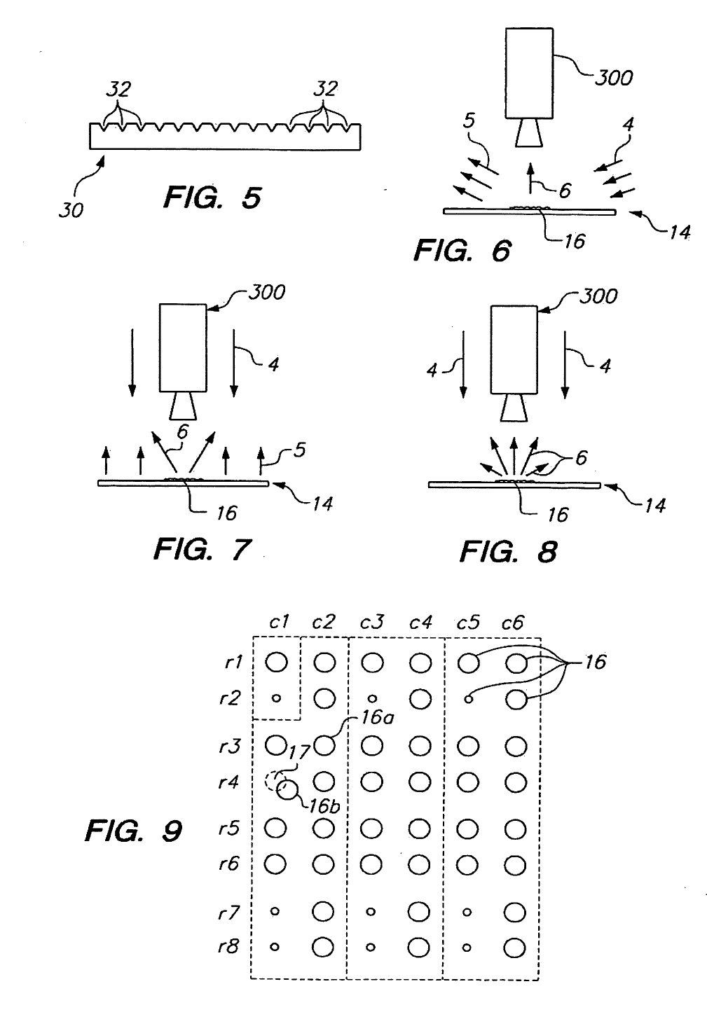

Referring to FIG. 4 the apparatus includes a substrate station 20 on which can be mounted a substrate 14. In FIG. 4 a mounted substrate is identified as substrate 14a, while a substrate which was previously mounted on substrate station 20 is identified as substrate 14b (both of these being generically identified as a substrate 14, and substrate 14b having been cut as mentioned below). Substrate station 20 can include a vacuum chuck connected to a suitable vacuum source (not shown) to retain a substrate 14 without exerting too much pressure thereon, since substrate 14 is often made of glass. A load station 30 is spaced apart from substrate station 20. Load station 30 can be of any construction with regions which can retain small volumes of different fluids for loading into head 210. For example, it may be a glass surface with different hydrophobic and hydrophilic regions to retain different drops thereon in the hydrophilic regions. Alternatively, the flexible microtitre plate described in U.S. patent application “Method and Apparatus for Liquid Transfer”, Ser. No. 09/183,604 could be used. In the drawings load station 30 has an upper surface with small notches 32 to assist in retaining multiple individual drops of a biopolymer fluid on that surface. The number of notches 32 or other regions for retaining drops of different fluids, is at least equal to (and can be greater than) the number of reservoir chambers in a printer head 210, and are spaced to align with orifices 214 in head 210.

A dispensing head 210 is retained by a head retainer 208. Head 210 can be positioned to face any one of loading station 30 or substrate station 20 by a positioning system. The positioning system includes a carriage 62 connected to each of the foregoing stations, a transporter 60 controlled by processor 140 through line 66, and a second transporter 100 controlled by processor 140 through line 106. Transporter 60 and carriage 62 are used execute one axis positioning of either of the stations 20 or 30, facing the dispensing head 210 by moving them in the direction of arrow 63, while transporter 100 is used to provide two axis adjustment of the position of head 210 in a vertical direction 202 or in the direction 204. Further, once substrate station 20 has been positioned facing head 210, the positioning will be used to scan head 208 across a mounted substrate 14, typically line by line (although other scanning configurations could be used). However, it will be appreciated that both transporters 60 and 100, or either one of them, with suitable construction, can be used to perform any necessary positioning (including the foregoing scanning) of head 210 with respect to any of the stations. Thus, when the present application recites “positioning” one element (such as head 210) in relation to another element (such as one of the stations 20, or 30) it will be understood that any required moving can be accomplished by moving either element or a combination of both of them.

Head retainer 208, and hence head 210, may communicate with a source of purging fluid (not shown) and suitable controlled pressure sources. Furthermore, a purging station and a cleaning station may be provided to clean both inside and outside head 210. Such features and their operation are described, for example, in U.S. patent applications entitled “FABRICATING BIOPOLYMER ARRAYS” by M. Caren et al., Attorney Docket No. 10990640 filed on the same day as the present application, and “PREPARATION OF BIOPOLYMER ARRAYS” by A. Schleifer et al., Attorney Docket No. 10990490 filed on the same day as the present application, and both assigned to the same assignee as the present application. Those references and all other references cited in the present application, are incorporated herein by reference. Head 210 may be of a type commonly used in an ink jet type of printer and may, for example, have one hundred fifty drop dispensing orifices in each of two parallel rows, six chambers for holding polynucleotide solution communicating with the three hundred orifices, and three hundred ejectors which are positioned in the chambers opposite a corresponding orifice. Each ejector is in the form of an electrical resistor operating as a heating element under control of processor 140 (although piezoelectric elements could be used instead). Each orifice with its associated ejector and portion of the chamber, defines a corresponding pulse jet. Thus, there are three hundred pulse jets in this configuration, although it will be appreciated that head 210 could, for example, have more or less pulse jets as desired (for example, at least ten or at least one hundred pulse jets). In this manner, application of a single electric pulse to an ejector causes a droplet to me dispensed from a corresponding orifice. In the foregoing configuration, typically about twenty orifices in each group of six reservoirs (many of the orifices are unused and are plugged with glue), will be dispensing the same fluid. Certain elements of the head 210 can be adapted from parts of a commercially available thermal inkjet print head device available from Hewlett-Packard Co. as part no. HP51645A. The foregoing head 210 and other suitable dispensing head designs are described in more detail in U.S. patent application entitled “A MULTIPLE RESERVOIR INK JET DEVICE FOR THE FABRICATION OF BIOMOLECULAR ARRAYS” Ser. No. 09/150,507 filed Sep. 9, 1998.

As is well known in the ink jet print art, the amount of fluid that is expelled in a single activation event of a pulse jet, can be controlled by changing one or more of a number of parameters, including the orifice diameter, the orifice length (thickness of the orifice member at the orifice), the size of the deposition chamber, and the size of the heating element, among others. The amount of fluid that is expelled during a single activation event is generally in the range about 0.1 to 1000 pL, usually about 0.5 to 500 pL and more usually about 1.0 to 250 pL. A typical velocity at which the fluid is expelled from the chamber is more than about 1 m/s, usually more than about 10 m/s, and may be as great as about 20 m/s or greater. As will be appreciated, if the orifice is in motion with respect to the receiving surface at the time an ejector is activated, the actual site of deposition of the material will not be the location that is at the moment of activation in a line-of-sight relation to the orifice, but will be a location that is predictable for the given distances and velocities.

The sizes of the spots can have widths (that is, diameter, for a round spot) in the range from a minimum of about 10 μm to a maximum of about 1.0 cm. In embodiments where very small spot sizes or feature sizes are desired, material can be deposited according to the invention in small spots whose width is in the range about 1.0 μm to 1.0 mm, usually about 5.0 μm to 500 μm, and more usually about 10 μm to 200 μm.

The apparatus further includes an inspection station having an imaging system which includes a camera 300 to capture one or more images of a substrate 14 on substrate station 20 and on which the deposit droplets have dried to form spots. Camera 300 is mounted for movement with head retainer 208 (and hence head 300) to facilitate image capture across the entire substrate 14 although a suitable camera 300 could be located in a fixed position if desired. However, since high resolution images are required from camera 300, and since a typical substrate may be about 12″ by 12″, camera 300 will not likely be able to yield images of the required resolution of all arrays 12 on a given substrate 14 simultaneously. Thus, precision movement of camera 300 will be required. Mounting camera 300 for movement with head 210 takes advantage of the precision movement already provided by transporter 100. Of course, the light sensor of a camera could potentially be mounted elsewhere, with a light receiving element (such as a mirror) mounted for movement with head 210 and arranged to direct light to the sensor (using other moving and/or stationary mirrors, for example). Any suitable analog or digital image capture device (including a line by line scanner) can be used as camera 300, although if an analog camera is used processor 300 should include a suitable analog/digital converter, and further more than one camera can be used if desired. A writer in the form of disk drive 320 is also provided along with a printer 350, display 310, speaker 314, and operator input device 312. Writer 320 may be an optical or magnetic writer (for example, a CD or disk drive) capable of writing onto a portable storage medium 324 (for example, an optical or magnetic disk). Operator input device 312 may, for example, be a keyboard, mouse, or the like. Processor 140 has access to a memory 141, and controls print head 210 (specifically, the activation of the ejectors therein), operation of the positioning system, operation of each jet in print head 210, capture of images from camera 300, and operation of writer 320, printer 350, display 310 and speaker 314. Memory 141 may be any suitable device in which processor 140 can store and retrieve data, such as magnetic, optical, or solid state storage devices (including magnetic or optical disks or tape or RAM, or any other suitable device). Processor 140 may include a general purpose digital microprocessor suitably programmed to execute all of the steps required by the present invention, or any hardware or software combination which will perform the required functions.

Substrate 14 may have any desired dimension. However, camera 300 will have to have sufficient resolution and to permit it to distinguish and observe each spot of an array. Movement of camera 300 with head retainer 208 facilitates it scanning over the entire substrate 14 and capturing multiple images with sufficient resolution such that a good image of each spot 16 of each array 12 is obtained. Camera 300 should have a resolution that provides a pixel size of about 1 to 100 micrometers and more typically about 4 to 10 micrometers.

Various configurations for camera 300 and an associated light source (not shown) may be used, as shown in FIGS. 6-8. For example, in FIG. 6 the light source provides input light 4 at an angle to substrate 14. The advantage of this configuration is the glass substrate 14 will appear dark to camera 300 since reflected light 5 is reflected from a surface of substrate 14 at the same angle. However, spot 16, and particularly dried salt crystals therein, scatter some input light 4 in the form of scattered light 6 which is directed toward camera 300. This allows processor 140 to acquire a high contrast image from camera 300. An alternative configuration is illustrated in FIG. 7. In the case of FIG. 7 input light 4 is perpendicular to substrate 14. Reflected light 5 from the surface of substrate 14 is directed straight back toward the light source, giving a very bright image to camera 300 from uncovered regions of substrate 14. However, dried spots 16 (and particularly dried salt crystals therein) result in scattered light 6 such that spot 16 will appear dark to camera 300. As in the configuration of FIG. 6, the configuration of FIG. 7 yields a high contrast image. The amount of any particular type of salt that may be used to enhance visibility of dried polynucleotide containing spots over dried spots not containing polynucleotide (but otherwise the same), can readily be determined by experimentation by comparing images of dried spots containing various concentrations of a salt of interest and the polynucleotide, with those of dried spots of the same composition except in which the polynucleotide is absent. It will also be appreciated that while it is preferred to use a salt for the reasons discussed below, other components which will scatter light in the dried spots, can be used instead of salt in any of the foregoing configurations.

A third configuration is illustrated in FIG. 8. In this configuration input light 4 is directed perpendicular toward substrate 14 as in FIG. 7. However, in this case the polynucleotide fluid has been provided with a fluorescent dye such that each spot 16 provides light 6 back to camera 300 which is at a different wavelength from the excitation input light 4. Camera 300 can use a filter to detect only light of the wavelength from fluorescing spots 16. The configuration of FIG. 8 has the advantage that spots 16 are readily detected even without the presence of salt crystals (that is, this configuration does not rely upon salt in the polynucleotide solution). Furthermore, in the configuration of FIG. 8 spots 16 of an array 12 can be imaged after exposure to a sample and immediately before scanning for the observed binding pattern. A user can then use the resulting information to discard or correct the results.

Operation of the apparatus of FIG. 4 in accordance with a method of the present invention, will now be described. First, it will be assumed that memory 141 holds an initial drop dispensing pattern for operating and coordinating scanning movement of head 210, in order to deposit spots 16 of different polynucleotides in a target pattern (which includes target locations and dimension for each spot). This initial drop dispensing pattern includes instructions for which polynucleotide solution is to be loaded in each pulse jet (that is, the “loading pattern”). This initial drop dispensing pattern is based upon the target spot pattern and can have either been input from an appropriate source (such as a portable magnetic or optical medium, or from a remote server), or may have been determined by processor 140 based upon the target spot pattern and the pulse jet configuration of head 210. Further, it will be assumed that drops of different biomonomer or biopolymer containing fluids (or other fluids) have been placed at respective regions of loading station 30 (such as the wells of the titer plate mentioned previously, or notches 32). This placement can be accomplished by manual or automated pipetting, or spotting of drops onto loading station 30 using glass rods, which are of a volume required to load all of the pulse jets. The placement pattern on notches 32 can be determined from the operator's knowledge or determined by processor 140 which could control an automated spotting system or could provide an operator with appropriate instructions on display 310 in the case of manual spotting. Operation of the following sequences are controlled by processor 140, following initial operator activation, unless a contrary indication appears.

For any given substrate 14, the operation is basically follows: (i) load head 210 with a first set of polynucleotide containing solutions (for example, a given head may be able to hold n different members); (ii) dispense droplets from head 210 onto substrate 14 or a set of substrates in a manner which is expected to provide the target pattern for the first set on each of multiple arrays; and (iii) repeat the foregoing sequence starting at step (i) with a second set and subsequent sets of polynucleotide containing solutions, until all required solutions have been dispensed onto substrate 14 (for example, if each array has m·n members, the sequence will be repeated m times). Inspection by capturing one or more images and performing the comparison, can be carried out at alternate or multiple times in the foregoing procedure, as desired. For example, an inspection could be performed on after step (ii) in each cycle. Preferably, all arrays on a given substrate 14 have been inspected before shipping to an end user. The foregoing steps are discussed in more detail below.

During the loading sequence of head 210, processor 140 directs the positioning system to position head 210 facing loading station 30 with the orifices aligned, facing, and adjacent to appropriate respective drops on loading station 30. As previously mentioned, during any positioning operation head 210 can be positioned to face the required station, by movement along one axis by transporter 60 and by movement along the other two axes by transporter 100. Processor 140 controls pressure within head 210 to load each polynucleotide solution into the chambers in the head by drawing it through the orifices. Such a technique is described in more detail in U.S. patent application entitled “FABRICATING BIOPOLYMER ARRAYS”, Attorney Docket No. 10990640, referenced above.

Substrate 14 is loaded onto substrate station 20 either manually by an operator, or optionally by a suitable automated driver (not shown) controlled, for example, by processor 140.

The deposition sequence is then initiated to deposit the desired arrays of polynucleotide containing fluid droplets on the substrate to provide dried drops on the substrate according to the target pattern each with respective target locations and dimensions. In this sequence processor 140 causes the positioning system to position head 210 facing substrate station 20, and particularly the mounted substrate 14, and with head 210 at an appropriate distance from substrate 14. Processor 140 then causes the positioning system to scan head 210 across substrate 14 line by line (or in some other desired pattern), while coordinating activation of the ejectors in head 210 so as to dispense droplets in accordance with the target pattern. If necessary or desired, processor 140 can repeat the load and dispensing sequences one or more times until head 210 has dispensed droplets in accordance with the target pattern for all arrays 12 to be formed on substrate 14. The number of spots in any one array 12 can, for example, be at least ten, at least one hundred, at least one thousand, or even at least one hundred thousand.

At this point the droplet dispensing sequence is complete. One or more images of all of the actual array patterns are then captured by camera 300 and processor 140 after a sufficient time has passed such that any droplets deposited by the deposition system will have dried. A typical value for the foregoing elapsed time may be at least about one second or even at least about one minute. This time can be measured by processor 140 knowing when droplet deposition was completed at deposition station 20. If during the deposition sequence all droplets were correctly deposited in accordance with the initial deposition pattern and dried without any further movement, they would yield the target array patterns of polynucleotide spots. In practice though, the actual spot patterns may be different from the target patterns due to factors such as those discussed above. Therefore, after the drying time has elapsed processor 140 captures the one or more images of the actual patterns on substrate 14b. It should be noted here that camera 300 or other imaging device, may be continuously viewing substrate 14b or the absence thereof. By “capturing” an image in this context is referenced only that processor 140 now obtains an image from camera 300 or other imaging device, for analysis (for example, after the predetermined drying time has elapsed, processor 140 then may select a single frame from camera 300 for use). Alternatively, after the predetermined drying time has elapsed, processor 140 could signal camera 300 to then capture a single frame which processor 140 uses for analysis, as described further below. The captured image can be stored by processor 140 in memory 141.

Processor 140 then compares the actual spot pattern contained within the captured image, with the target pattern, both patterns now being present in memory 141. This pattern comparison can particularly include spot location and dimensions (such as the area of each spot). Processor 140 generates a signal from the results of the comparison. The signal may, for example, be a value representing the differences in position of each target spot versus that of the corresponding actual spot (which could be measured by the degree of overlap of the target and actual spot positions). The signal may further include a difference in actual and target spot sizes. The value of each of these location and dimension comparison signals can be tested against predetermined tolerances. When an actual spot has all comparison values within the tolerances (for example, position and size values are within the tolerance) it will be considered acceptable without more (that is, it will be considered error free), and the results of the comparison need not be stored. When an actual spot has one comparison value beyond the tolerance it will be considered in error and an indication of the error stored in memory 141 in association with an identification of the particular array on substrate 14b. The stored error indication includes an identification of the spot location on the particular array and the type and magnitude of the error. For example, in addition to the spot identification, the error indication may identify that the particular spot is actually located at an identified position relative to other spots or a reference position on the substrate, or that the spot has an incorrect area of a determined value. It should be noted at this point that indications on spots considered acceptable may optionally also be stored, such that memory 141 contains a complete actual pattern (that is a “map”) of all actual spots of each array. In effect then, memory 141 will contain an error map for all spots, although this map may optionally also contain information on all spots considered acceptable.

A substrate such as 14b is then typically (but need not be) cut into a desired number of pieces by a cutter 150 (which may be manually or automatically operated), with separated sections each carrying one or more arrays (such as section 15) then being directed into respective packages (such as package 340) for delivery to a remote customer.

The above sequence can be repeated as desired for multiple substrates 14 in turn. During any sequence, after capturing an image of an actual pattern on each array on a substrate 14, and comparing the actual spot pattern with the target pattern (in particular actual spot locations or dimensions with target locations or dimensions), processor 140 may respond in any of the ways discussed below.

Processor 140 can be programmed to respond in any of a number of ways to errors. This response can either be pre-programmed into processor 140 as the way it will respond, or a number of different response options can be presented to an operator on display 310 to select an operator desired one by means of input device 312. In a particular implementation, processor 140 can operate with first and second level error tests. First level errors can be considered spot errors which fall within the predetermined tolerances. Second level errors can be considered to occur when a predetermined number of spots in an array (such as one or more or ten or more) have errors exceeding one or more tolerances by a predetermined amount. For example, second level errors may be considered to occur when a large number of spots in an array have any errors, or when a smaller number of spots have errors which exceed the tolerance by a predetermined amount. In this implementation first level errors can be ones which are considered “acceptable” in that the associated array (or at least some arrays on a same substrate) is still useful, while second level errors are considered so severe as to require the array not be used (that is, that it be rejected). In the case of first level errors for one or more arrays on a substrate 14, processor 140 can cause an identification of these errors to be written by drive 320 onto portable storage medium 324. Alternatively or additionally, an identification of these errors can be written by printer 350 onto a medium in the form of a paper sheet 354 in either machine readable characters (for example, bar codes) or in human readable characters (for example, alphanumeric or other characters). These identifications may contain the actual data specifying the spot error types and their magnitudes. Alternatively, these identifications may be unique arbitrary identifications generated by processor 140 and stored in memory 141 in association with the actual error map, so that the actual error map can be retrieved (such as from a remote computer over a communication line, as mentioned below) from memory 141 by an end user of the arrays using the identifications. The medium on which the identifications are written, can be physically associated with the corresponding arrays on a section such as section 15, by packaging each array and any such medium together in a single package 340. Other ways of accomplishing this physical association to provide the user with, in effect, a kit containing an array and one or both of such mediums, can of course be used. For example, paper sheet 354 may be adhesive to allow its attachment to the back of a substrate 14. Where a substrate 14 provided to a user carries multiple arrays 12, the medium will carry an identification of the array with which it is associated (for example, by reference to an array location or number).

On a second level error, processor 140 can be programmed to direct the associated array be rejected so that it cannot be used by an end user. This can be done in a number of ways. For example, processor 140 can direct an operator to manually reject such an identified array by displaying instructions on display 310 or providing them over speaker 314. The operator can reject the array by, for example, disposing of an entire substrate such as substrate 14b, bearing the rejected array. Alternatively, if automated equipment is used to handle substrates 14 and direct them into respective packages such as package 340, processor 140 can direct an individual rejected array or an entire substrate 14 carrying such an array into a trash bin. If individual arrays and respective portions of substrate 14 are separated (such as by cutting by cutter 150) into sections (such as section 15) carrying one or more arrays, processor 14 stores an identification of any arrays having second level errors and tracks their position and, following separation, directs the pieces carrying those arrays into a trash bin.

In addition on a second level error or, if desired by an operator (such as by selection on input device 312 based on a selection screen shown on display 310) on any selected error, operation of the apparatus can be automatically halted and a visible or audible operator alert generated on display 310 or speaker 314. This alert can include an identification of the error type and its magnitude.

When multiple errors occur in the same or different arrays, processor 140 may be able to evaluate the cause of the error. Processor 140 can accomplish this evaluation using the actual spot pattern, particularly when compared with the pattern in which head 210 was loaded with polynucleotide containing fluids. This process can be better understood by reference to FIG. 9. The following convention will be used to identify particular spots in each of FIGS. 9 through 13. In particular each array portion illustrated is assigned row numbers (beginning with “r”) and column numbers (beginning with “c”). An identification of any one spot will include the FIG. number followed by the row and column number. For example, spot 16a in FIG. 9 is identified as 9r3c2.

Referring to FIG. 9, the solid circles of different sizes represent actual dried spots 16 as might be seen in an image captured by camera 300. This array portion was formed from drops deposited by a hypothetical head having two rows of eight pulse jets each, in a single pass from left to right as viewed in FIG. 9. Thus, in this simple case, columns c1 and c2 were formed by deposition from corresponding pulse jets in such a head. Similarly, columns c3 and c4 were formed by subsequent depositions from those same corresponding pulse jets after movement of the head to the right in FIG. 9. Further movement and operation of the head deposited drops forming spots 16 in columns c5 and c6. This head was previously loaded in a pattern such that each pair of adjacent pulse jets in a columnar direction in FIG. 9, had the same cDNA solution. Thus, 9r1c1 and 9r2c1 should have the same cDNA. Similarly the members of the following pairs, for example, will each have the same cDNA (although each pair may have cDNA different from any other pair): 9r5c1/9r6c1; 9r7c1/9r8c1; 9r1c2/9r2c2; 9r3c2/9r4c2; 9r5c2/9r6c2; 9r5c5/9r6c5; 9r7c5/9r8c5; and so on.

In FIG. 9, all of the spots 16 are in their target position forming a regular rectangular array, with the exception of spot 9r4c1 (also identified as spot 16b). Processor 140, by comparing the actual dried spot pattern with the target pattern, will determine that spot 9r4c1 is displaced from its target position 17 (indicated by the broken line circle in FIG. 9), and can calculate the magnitude (including direction) of the displacement. This displacement will be assumed to be a displacement which exceeds a predetermined position tolerance, and so spot 16b has a displacement error. On the other hand, a number of the actual spots 16 (such as spots 9r2c1, 9r7c1, 9r8c1, 9r2c2, and others) have a total area which is substantially less than the target area (as represented by, for example, spot 16a). These areas will be assumed to be different from a target area by an amount which exceeds a predetermined area tolerance, and so such spots have area errors.

Processor 140 can now attempt to evaluate the cause of the errors by examining the error pattern in the dried spots along with the load pattern of the head, as needed. For example, spot 9r4c1 was deposited by the same pulse jet as spots r4c3 and r4c5 which do not have any errors. Thus, based on this portion of the array in any event (and a larger portion may provide an alternate indication) it can probably safely be assumed that the error in spot 9r4c1 was caused by a random factor (for example, a vibration). On the other hand, each of spots 9r2c1, 9r2c3 and 9r2c5 exhibit an area error. This could be a pulse jet error or, as explained below, the small spot size could have been caused by lack of DNA even though the pulse jet was functioning normally. However, since spots 9r1c1, 9r1c3 and 9r1c5 do not exhibit any size error and they formed from the same polynucleotide solution dispersed from an adjacent pulse jet, it can safely be assumed that the error was not in that solution but in the single pulse jet responsible for forming spots 9r1c1, 9r1c3 and 9r1c5. Turning to spot pairs 9r7c1/9r8c1, 9r7c3/9r8c3, and 9r7c5/9r8c5, all of these spots have an area error. As already mentioned, this could be caused by error in the cDNA solutions or in the responsible pulse jets. However, the likelihood of two adjacent pulse jets failing is probably slight, such that the most likely cause of these spot errors is probably an error in the same cDNA solutions. The most likely causes of any of the spot errors determined from the foregoing evaluations, can be reported on display 310 or speaker 314 as potential errors resulting from those causes (for example, a potential polynucleotide containing fluid error, or potential pulse jet error), whether or not any one or more errors is treated as second level error.

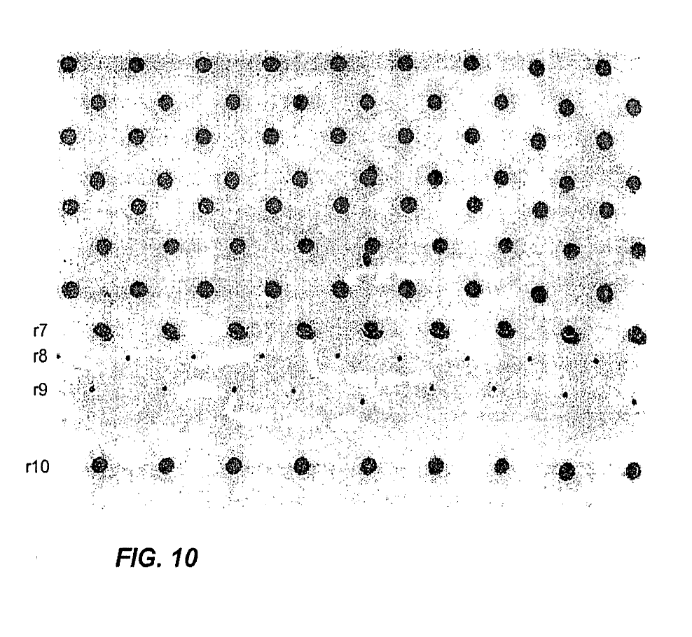

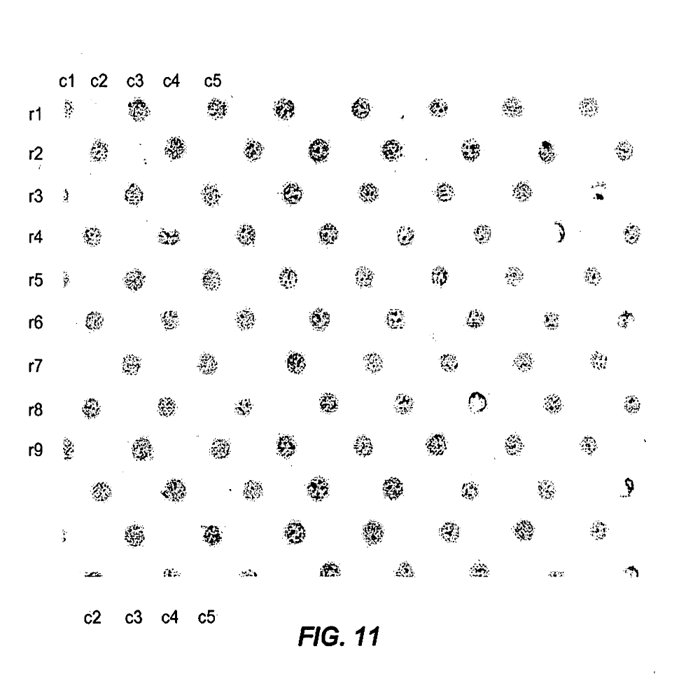

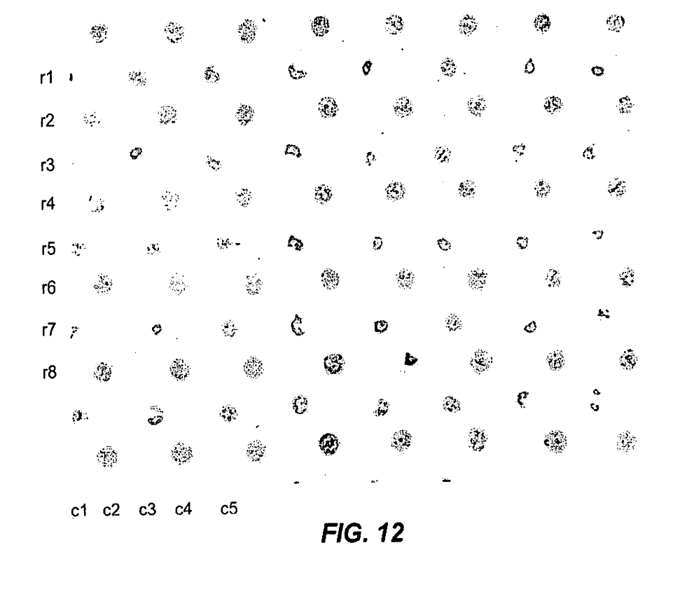

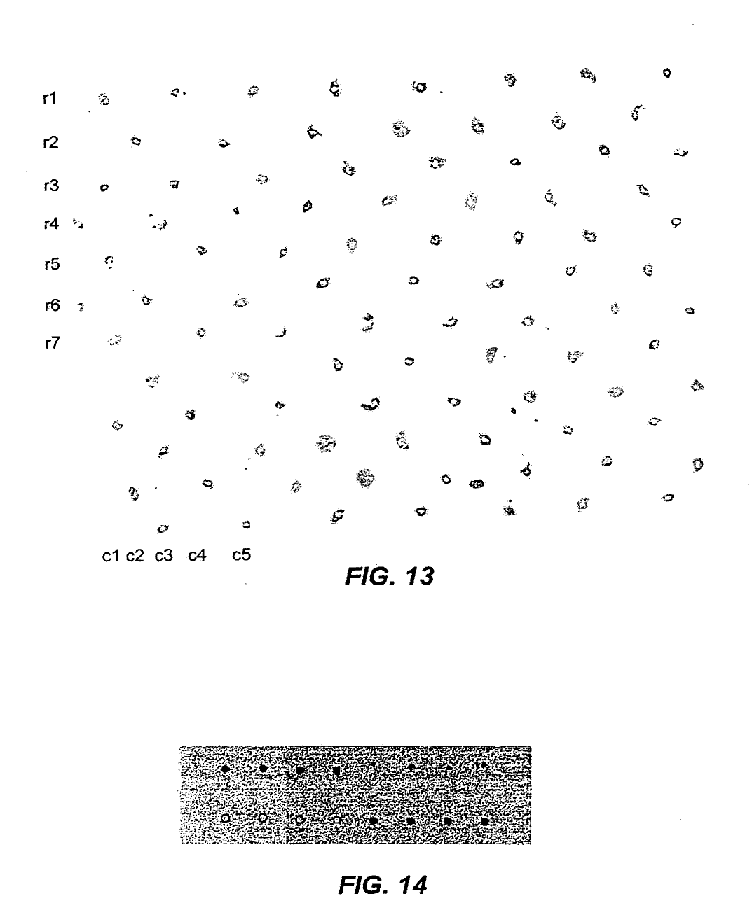

Referring now to FIGS. 10-13 these illustrate that a failure in a polynucleotide solution (specifically a cDNA solution) can show up as a significantly reduced spot area, other factors remaining the same. In particular, to obtain the solutions used in FIG. 10, an “SSC” buffer solution can be made by dissolving 175.3 g of NaCl and 88.2 g of sodium citrate in 800 ml of water. The pH is adjusted to 7.0 with a few drops of a 10N NaOH solution. The volume is then adjusted to 1 liter with water, and the resulting solution diluted with water to 1/20 the concentration. For the solutions used to form the spots in rows 1-7, a cDNA concentration was provided in SSC buffer of 0.25 μg/μl. Each of rows 1-7, and 10 contained respective different cDNAs. In the case of rows 8 and 9 the same SSC buffer solution was used without the addition of any DNA. The same volume of solution was deposited onto a glass substrate such that a circular spot size of about 70 μm in diameter was obtained for all spots containing cDNA. On the other hand, the drops in rows 8 and 9 not containing any DNA are significantly smaller in area. Similarly, in all of FIGS. 11-13 the same DNA was used in SSC solution but at different concentrations. In particular, in FIG. 11, each odd rows (such as r7) used a cDNA at respective concentrations of 0.25 μg/μl while odd rows (such as r7) used a concentration of 0.025 μg/μl. Similarly, in FIG. 12 even rows (such as r6) used a DNA concentration of 0.25 μg/μl while odd rows (such as r5) used a DNA concentration of 0.001 μg/μl. In FIG. 13 even rows (such as r4) used a DNA concnetration of 0.005 μg/μl while odd rows (such as r5) used a DNA concnetration of μg/μl. Note that at the same concentrations, spot size for different cDNAs does not vary significantly. Also, while a single order of magnitude change in concentration does not reliably decrease spot area, as seen in FIG. 10 much larger drops in concentration do result in significantly decreased spot size. Thus, significant errors in cDNA concentration (such as when no cDNA is present due to operator error or amplification reaction failure) can be detected in the foregoing salt solution.

FIG. 14 illustrates dried spots on an array prepared in the same manner as those of FIGS. 10-13. The first four spots on the left of the first row were prepared using a first DNA at a concentration of 0.125 μg/μl in SSC. The last four spots on the right of the first row were prepared in the same manner as the first four but with no DNA (that is, with SSC solution only). The first four spots on the left side of the second row used the first DNA at a concentration of 0.50 μg/μl and with the SSC salts omitted. The last four spots on the right of the second row used a second DNA at a concentration of 0.125 μg/μl. As is apparent from FIG. 14, the presence of the salts in the dried spots considerably enhanced the visibility of the DNA.

In some cases, processor 140 may not only be able to evaluate the source of an error, but may also be able to compensate for the errors. For example, in the case of a likely pulse jet malfunction, processor 140 can alter the initial drop dispensing pattern to form a new dispensing pattern in which use of a suspect pulse jet is avoided. This new dispensing pattern is then stored in memory 141 by processor 140 to become a new initial dispensing pattern, which is followed by processor 140 in subsequent drop dispensing for arrays of the same target pattern until a further error pattern indicates another potential source of error (in which case the drop dispensing pattern can again be altered). Depending upon the array being formed and the dispensing head pulse jet configuration, a new dispensing pattern may require one or more additional passes of the head over the substrate than did the initial pattern.

When a remote customer receives a package such as package 340, the received section 15 may be exposed to a sample (which may be labeled) in a known manner under appropriate conditions (such as hybridizing conditions). The resulting observed binding pattern may be determined by a reader 162. Reader 162 may, for example, be able to detect the fluorescence of a label in a known manner. It will be appreciated though, that if a first fluorescent compound is used in the polynucleotide containing fluid during deposition, such that camera 300 and processor 140 can identify the actual spot pattern based upon first compound fluorescence, any fluorescent label should have a different spectral emission than the first fluorescent compound (and preferably they do not overlap to any substantial extent) to avoid reader 162 detecting fluorescence of the first fluorescent compound rather than the label. In this circumstance, reader 162 should of course have a detector which can specifically detect the fluorescence of the label.

A reader 160 is capable of reading either the identification on portable storage medium 324 or the identification on paper 354. In the case where the identification on paper 354 is in human readable characters, reader 160 may simply be an operator input device. When the identification read by reader 160 contains the actual error indication data in the form of the error map, reader 162 may use this data to either modify its initial determination of the observed binding pattern, or to alter the results of the determination based on the received error indications of the error pattern. For example, where the error indication is that a spot 16 is defective and should not be used, reader 162 may modify its initial determination of the observed binding pattern by skipping any determination of fluorescence from that spot. Alternatively, as mentioned above the identification read by reader 160 may be a unique arbitrary identification generated by processor 140 and stored in memory 141 in association with the actual error map, as mentioned above. In this case, the error map may be retrieved from remote memory 141 by a communication module 164 acting in conjunction with a communication module 144 and processor 140 through a communication channel (such as a network, including the Internet). In this configuration processor 140 acts as a remote server. Once retrieved, the error map can be utilized by reader 162 to control initial reading of a section 15 or to correct the read data, as already mentioned.

Modifications in the particular embodiments described above are, of course, possible. For example, where a pattern of arrays is desired, any of a variety of geometries may be constructed other than the organized rows and columns of arrays 12 of FIG. 1. For example, arrays 12 can be arranged in a series of curvilinear rows across the substrate surface (for example, a series of concentric circles or semi-circles of spots), and the like. Similarly, the pattern of dried spots 16 may be varied from the organized rows and columns of spots in FIG. 2 to include, for example, a series of curvilinear rows across the substrate surface(for example, a series of concentric circles or semi-circles of spots), and the like.

The present methods and apparatus may be used to deposit biopolymers or other moieties on surfaces of any of a variety of different substrates, including both flexible and rigid substrates. Preferred materials provide physical support for the deposited material and endure the conditions of the deposition process and of any subsequent treatment or handling or processing that may be encountered in the use of the particular array. The array substrate may take any of a variety of configurations ranging from simple to complex. Thus, the substrate could have generally planar form, as for example a slide or plate configuration, such as a rectangular or square or disc. In many embodiments, the substrate will be shaped generally as a rectangular solid, having a length in the range about 4 mm to 200 mm, usually about 4 mm to 150 mm, more usually about 4 mm to 125 mm; a width in the range about 4 mm to 200 mm, usually about 4 mm to 120 mm and more usually about 4 mm to 80 mm; and a thickness in the range about 0.01 mm to 5.0 mm, usually from about 0.1 mm to 2 mm and more usually from about 0.2 to 1 mm. The configuration of the array may be selected according to manufacturing, handling, and use considerations.

The substrates may be fabricated from any of a variety of materials. In certain embodiments, such as for example where production of binding pair arrays for use in research and related applications is desired, the materials from which the substrate may be fabricated should ideally exhibit a low level of non-specific binding during hybridization events. In many situations, it will also be preferable to employ a material that is transparent to visible and/or UV light. For flexible substrates, materials of interest include: nylon, both modified and unmodified, nitrocellulose, polypropylene, and the like, where a nylon membrane, as well as derivatives thereof, may be particularly useful in this embodiment. For rigid substrates, specific materials of interest include: glass; plastics (for example, polytetrafluoroethylene, polypropylene, polystyrene, polycarbonate, and blends thereof, and the like); metals (for example, gold, platinum, and the like).

The substrate surface onto which the polynucleotide compositions or other moieties is deposited may be smooth or substantially planar, or have irregularities, such as depressions or elevations. The surface may be modified with one or more different layers of compounds that serve to modify the properties of the surface in a desirable manner. Such modification layers, when present, will generally range in thickness from a monomolecular thickness to about 1 mm, usually from a monomolecular thickness to about 0.1 mm and more usually from a monomolecular thickness to about 0.001 mm. Modification layers of interest include: inorganic and organic layers such as metals, metal oxides, polymers, small organic molecules and the like. Polymeric layers of interest include layers of: peptides, proteins, polynucleic acids or mimetics thereof (for example, peptide nucleic acids and the like); polysaccharides, phospholipids, polyurethanes, polyesters, polycarbonates, polyureas, polyamides, polyethyleneamines, polyarylene sulfides, polysiloxanes, polyimides, polyacetates, and the like, where the polymers may be hetero- or homopolymeric, and may or may not have separate functional moieties attached thereto (for example, conjugated),

Various modifications to the embodiments of the particular embodiments described above are, of course, possible. Accordingly, the present invention is not limited to the particular embodiments described in detail above.

What is claimed is:

1. A method of fabricating an array of biopolymers on a substrate, comprising:
(a) operating a deposition system to deposit fluids on the substrate in accordance with a target pattern to produce an actual pattern of deposited fluid droplets on said substrate, which fluids contain the biopolymers or biopolymer precursors;
(b) capturing an image of the actual pattern of the deposited fluids; and
(c) comparing the actual pattern with the target pattern.

2. A method according to claim 1, wherein the image of the actual pattern is captured before the deposited fluids dry.

3. A method according to claim 1, wherein the image of the actual pattern is captured after the deposited fluids dry.

4. A method according to claim 1, wherein the deposited droplets contain polynucleotides or precursors thereof.

5. A method according to claim 1, wherein the deposited droplets contain an amino acid containing polymer or precursors thereof.

6. A method according to claim 1, wherein the deposition system comprises a pin drop dispenser.

7. A method according to claim 1, wherein the deposition system comprises a capillary drop dispenser.

8. A method according to claim 1, wherein the deposition system comprises a pulse-jet drop dispenser.

9. A method according to claim 8, wherein said pulse-jet drop dispenser comprises a heating element.

10. A method according to claim 8, wherein said pulse-jet drop dispenser comprises a piezoelectric element.

11. A method according to claim 1, wherein the comparison includes a comparison of actual spot locations or dimensions with target locations or dimensions.

12. A method according to claim 1, additionally comprising generating a signal indicative of the result of the comparison.

13. A method according to claim 1, additionally comprising:
generating an error indication based on the comparison.

14. A method according to claim 13, wherein the deposition system is operated to fabricate multiple polynucleotide arrays, the method additionally comprising rejecting the array associated with an error indication or correcting a cause of the error.

15. A method according to claim 13, additionally comprising:
writing the error indication or an identification of the error indication, on a medium; and
physically associating the medium with the array;
wherein when an identification of the error indication is written on the medium, the method further comprising storing the identification in a memory in association with the error indication.

16. A method according to claim 13, wherein the polynucleotide deposition system includes a fluid dispensing head with multiple drop dispensers, the method additionally comprising:
when multiple error conditions are generated, evaluating if a same drop dispenser is responsible; and
when the evaluation indicates the same drop dispenser is responsible, generating a visible or audible operator alert which includes an indication of the responsible drop dispenser or altering the drop deposition pattern such that the responsible drop dispenser is not used.

17. A method according to claim 1, wherein the target and actual patterns are for a test print deposited prior to fabrication of the array.

18. A method according to claim 1, wherein the target and actual patterns are for the array.

19. A method of fabricating an array of biopolymers on a substrate, comprising:
(a) operating a deposition system to deposit fluid droplets on the substrate in accordance with a target pattern to produce an actual pattern of deposited fluid droplets on said substrate, which droplets contain the biopolymers or biopolymer precursors;
(b) capturing an image of the actual pattern of the deposited droplets; and
(c) saving the actual pattern in a memory.

20. A method according to claim 19, wherein the image of the actual pattern is captured before the deposited fluids dry.

21. A method according to claim 19, wherein the image of the actual pattern is captured after the deposited fluids dry.

22. A method according to claim 19, wherein the deposited droplets contain polynucleotides or precursors thereof.

23. A method according to claim 19, wherein the deposited droplets contain an amino acid containing polymer or precursors thereof.

24. A method according to claim 19, wherein the deposition system comprises a pin drop dispenser.

25. A method according to claim 19, wherein the deposition system comprises a capillary drop dispenser.

26. A method according to claim 19, wherein the deposition system comprises a pulse-jet drop dispenser.

27. A method according to claim 26, wherein said pulse-jet drop dispenser comprises a heating element.

28. A method according to claim 26, wherein said pulse-jet drop dispenser comprises a piezoelectric element.

29. A method according to claim 19, additionally comprising saving an identification in the memory in association with the actual pattern.

30. A method according to claim 29, additionally comprising writing the identification on a medium and physically associating that medium with the array.

31. A method according to claim 30, wherein the identification is attached to the substrate.

32. A method according to claim 29 additionally comprising communicating the actual pattern in response to receiving the identification.

33. A method according to claim 19, wherein the memory comprises a portable storage medium which is sent to a user with the array.

34. A method according to claim 33, wherein the portable storage medium is sent in a same package with the array.

35. A method according to claim 34, additionally comprising writing the identification on the portable storage medium.

36. A method of fabricating an array of biopolymers on a substrate, comprising:
(a) operating a deposition system to deposit fluid droplets on the substrate in accordance with a target pattern to produce an actual pattern of deposited fluid droplets on said substrate, which droplets contain the biopolymers or biopolymer precursors;
(b) capturing an image of the actual pattern;
(c) comparing the actual pattern with the target pattern;
(e) when the results of one or more comparisons for an array exceed a predetermined tolerance, generating an error indication associated with the array; and
(f) writing the error indication or an identification of the error indication, on a medium; and
(g) physically associating the medium with the array;
wherein when an identification of the error indication is written on the medium, the method further comprising storing the identification in the memory in association with the error indication.

37. A method according to claim 36, wherein the image of the actual pattern is captured before the deposited fluids dry.

38. A method according to claim 36, wherein the image of the actual pattern is captured after the deposited fluids dry.

39. A method according to claim 36, wherein the deposited droplets contain polynucleotides or precursors thereof.

40. A method according to claim 36, wherein the deposited droplets contain an amino acid containing polymer or precursors thereof.

41. A method according to claim 36, wherein the deposition system comprises a pin drop dispenser.

42. A method according to claim 36, wherein the deposition system comprises a capillary drop dispenser.

43. A method according to claim 36, wherein the deposition system comprises a pulse-jet drop dispenser.

44. A method according to claim 43, wherein said pulse-jet drop dispenser comprises a heating element.

45. A method according to claim 43, wherein said pulse-jet drop dispenser comprises a piezoelectric element.

46. A method according to claim 36, wherein the identification is attached to the substrate.

47. A method according to claim 36, additionally comprising communicating the actual pattern in response to receiving the identification.

48. A method according to claim 36, wherein the memory comprises a portable storage medium which is sent to a user with the array.

49. A method according to claim 48, wherein the portable storage medium is sent in a same package with the array.

50. A method according to claim 49, additionally comprising writing the identification on the portable storage medium.
